(19) United States
(12) Patent Application Publication
(10) Pub. No.: US 2026/0294389 A1
(43) Pub. Date: Oct. 1, 2026

(54) Wireless Pullback Distance Sensor For IVUS Systems

(71) Applicant: NuevoSono Inc., Santa Clara, CA (US)

(72) Inventors: Sean Madden, Arlington, MA (US); Reza Kassayan, Atherton, CA (US)

(21) Appl. No.: 19/480,637

(22) PCT Filed: May 3, 2024
(86) PCT No.: PCT/US2024/027760

§ 371 Date: Oct. 31, 2025

Related U.S. Application Data
(60) Provisional application No. 63/464,338, filed on May 5, 2023.

(51) Int. Cl. A61B 8/12; A61B 8/00; A61B 8/08

(52) U.S. Cl. CPC A61B 8/12; A61B 8/0841; A61B 8/4427; A61B 8/4433; A61B 8/445; A61B 8/565

(57) ABSTRACT

The present invention relates to intravascular techniques for which pullback distance measurement is important, such as intravascular ultrasound (IVUS) imaging. The invention describes hardware, circuit architectures and methods for wireless transmission of data related to pullback distance of a catheter that is introduced into a vessel.

### RELATED APPLICATION DATA

This application claims the benefit of priority of U.S. Provisional Patent Application Ser. No. 63/464,338, filed May 5, 2023, and titled “Wireless Pullback Distance Sensor”, which is incorporated by reference herein in its entirety.

### FIELD OF THE DISCLOSURE

The present disclosure relates to intravascular ultrasound (IVUS) and, more specifically, to systems and methods for measurement of the pullback distance of IVUS catheters.

### BACKGROUND

Intravascular ultrasound (IVUS) is an imaging method for assessment of vascular pathologies and the guidance of vascular interventions. IVUS is of particular value to coronary percutaneous interventions (PCI) where assessment of plaques and procedural guidance can be difficult when utilizing coronary angiography alone. Coronary IVUS was first made commercially available in the early 1990s. The technology has expectedly steadily improved, and substantial clinical evidence for the benefit of IVUS for PCI has accumulated. U.S. Pat. No. 8,864,674 to Corl, entitled “Circuit Architectures and Electrical Interfaces for Rotational Intravascular Ultrasound (IVUS) Devices” describes an example of a conventional IVUS system and is incorporated by reference herein in its entirety. The additional imaging information provided by IVUS, such as identification of the presence and extent of plaque, more accurate vessel sizing, measurement of the external elastic membrane (EEM), and assessment of stent deployment has potential to improve the safety of stenting, including optimization of length of vessel to stent, selection of stent landing zones, and identification lesions at higher risk of distal embolization during stenting. The proven ability of IVUS to identify high-risk lesions can also facilitate implementation of strategies to prevent future coronary events. Clinical studies have shown that treatment decisions for individual patients are complex and often surrounded by uncertainty. The clinician is faced daily with challenging decisions regarding revascularization strategies (e.g., PCI versus bypass surgery) and important procedural issues regarding the likelihood of success of PCI (e.g., identification of which lesions to treat, length of vessel to treat, optimal stent deployment, risk of distal embolization and periprocedural myocardial infarction (MI), etc.).

While the overwhelming majority of PCI cases in the US are performed only under angiographic guidance, coronary angiography alone fails to provide adequate information for such complex decisions. Importantly, coronary angiography is necessarily only imaging the inner lumen of vessels, since the images are formed by the contrast briefly flowing through the arteries. Any information about the vessel wall (e.g., plaque burden) remains hidden. Dimensional accuracy is also limited, due to low spatial resolution and many sources of image artifacts (e.g., vessels twisting away from or towards the viewing detector). Angiography is notorious for high variability of interpretation for parameters that will quantitatively determine whether an intervention is even performed (e.g., degree of stenosis). Although a crucial tool to delineate the gross presence of disease and rough quantification of the degree of stenosis, angiography underestimates the magnitude of atherosclerotic burden, particularly in earlier stage of the disease during which positive vascular remodeling may allow normal-size lumen despite substantial plaque that has developed in the vascular wall. Importantly, angiography also has significant limitations in the precise measurement of plaque architecture and cannot provide data on plaque composition beyond a rough idea of degree of calcification. The selection and placement of drug-eluting coronary stents (DES) on the basis of angiographic information alone has been associated with increased complications and poorer outcomes compared to PCI guided by IVUS.

IVUS-guided PCI has been shown to reduce stent complications (dissection, stent thrombosis, or edge restenosis), geographical miss in stent placement (stent ends in disease or an otherwise non-optimal location), and incomplete stent expansion or poor apposition. As a comprehensive example of evidence, a meta-analysis encompassing 11 studies in 19,619 patients demonstrated the superiority of direct coronary imaging to facilitate PCI compared with angiography guidance. Another major study showed IVUS guidance changed clinicians' decisions in >75% cases, resulting in longer, more appropriately sized, and more effectively deployed stents. PCI performed with IVUS guidance has repeatedly been shown to be associated with a >50% relative risk reduction in the composite endpoint of cardiac death, myocardial infarction, and stent thrombosis.

Atherosclerotic lesions having characteristics indicating propensity to imminent rupture, so-called vulnerable plaques, are of particular interest to interventional cardiologists and are thought to be the cause of the majority of heart attacks. The ex vivo pathological analogue to the in vivo vulnerable plaque is a thin-cap fibroatheroma (TCFA), defined primarily by the presence of a large necrotic core and a thin (<65 μm) overlying fibrous cap. Thus, to begin to interpret the possible presence of a vulnerable plaque, an IVUS system must first have adequate contrast and tissue border differentiation (e.g., blood, lumen border, external elastic membrane, thrombus, calcium, etc.) to quantify the so-called plaque burden (size of plaque) to speculate that the plaque contains necrotic core, and then the resolution must be adequate (e.g., <50 um) to visualize regions of thin cap. Even the best current commercially available IVUS systems have only moderate image quality, which can limit the ability to identify vulnerable plaques. The same image quality parameters that are relevant to plaque characterization for prediction of a patient's long-term risk of a future event are also of immediate importance for at-patient procedural guidance (precise stent location relative to plaque location, stent sizing, confirmation of stent deployment success, or assessment of stent healing in a follow-up catheterization).

As previously noted, measurement of lesion length and related parameters that are defined by longitudinal position in the vessel are very important. There are two basic types of commercially available catheters—catheters with a single transducer element and a rotating and retractable core, and catheters with a multiplexed array of transducers at the tip. Single-transducer rotating-core catheters achieve a cross-sectional image frame by rotating at high speed (usually 1800 RPM or 30 frames per second) and create volumetric images by retracting the core while continuing to collect frames (a so-called “pullback”). Pullback length is tracked in the motor drive unit by digitization of the distance the core is retracted relative to the sheath. Pullback information is conveyed to the console and image processing code via a wired connection between the motor drive unit and the console. Catheters with a multiplexed array of transducers at the tip (“phased array” catheters) produce an image by a sequence of excitation and detection of the circularly arranged transducers to create a virtual rotation and corresponding frame. Volumetric images can be obtained by retracting the entire catheter from the vessel while continuing to acquire frames. Commercial pullback devices have notoriously had significant accuracy and variability issues.

### SUMMARY OF THE DISCLOSURE

In one implementation, the present disclosure is directed to a pullback distance sensor for intravascular ultrasound systems (IVUS). The sensor includes a housing defining a central opening configured to slidingly receive an IVUS catheter therethrough; a linear motion sensor disposed with the housing configured to detect motion of the IVUS catheter when moved through the housing central opening; and a wireless communications device disposed with the housing in communication with the linear motion sensor to receive and wirelessly transmit signals representing movement of the IVUS catheter detected by the linear motion sensor.

In another implementation, the present disclosure is directed to an intravascular ultrasound system (IVUS), which includes an IVUS patient interface module; an IVUS catheter extending from the patient interface module; an IVUS catheter introducer configured to receive the IVUS catheter; a pullback distance sensing unit configured to be disposed around the IVUS catheter and produce a wireless signal indicating pullback distance, wherein the wireless signal is received by an IVUS system controller.

In yet another implementation, the present disclosure is directed to a method of measuring pullback distance in an intravascular ultrasound system (IVUS), which includes placing a pullback distance sensing unit housing around an IVUS catheter; pulling the IVUS catheter through the pullback distance sensing unit; detecting a distance of travel of the IVUS catheter through the pullback distance sensing unit housing with at least a linear motion sensor disposed with the unit housing; and wirelessly transmitting a signal indicating the distance of travel from the pullback distance sensing unit housing to an IVUS system controller.

### BRIEF DESCRIPTION OF THE DRAWINGS

For the purpose of illustrating the disclosure, the drawings show aspects of one or more embodiments of the disclosure. However, it should be understood that the present disclosure is not limited to the precise arrangements and instrumentalities shown in the drawings, wherein:

FIG. 1 is a schematic diagram of an IVUS system according to the present disclosure.

FIG. 2 is a detailed view of a motion sensing and transmitting unit according to an embodiment of the present disclosure.

FIG. 3 is a schematic depiction of the operation of a motion sensing and transmitting unit according to an embodiment of the present disclosure.

FIG. 4 is a block diagram illustrating sensor communication according to an embodiment of the present disclosure.

FIG. 5 is a schematic end view of an alternative embodiment of a sensing unit according to the present disclosure.

### DETAILED DESCRIPTION

IVUS systems and methods are described herein including motion sensing and transmitting units configured to provide more precise pullback measurements with greater accuracy and repeatability. Embodiments described herein and shown in the attached figures include, but are not limited to, sensing of the pullback position of the catheter with a small, disposable accessory that connects to the introducer Luer-lock connector and transmits the information continuously and wirelessly back to the motor drive unit, wherein sensing can be one or more of purely optical (bounce light off the side of the catheter), a wheel in contact with the catheter that turns as the catheter moves longitudinally and has a slotted disk with optical measurement, and/or the catheter may have graduations printed on the side to improve accuracy of the sensor, and sensor circuitry and digitization may be done with an ASIC.

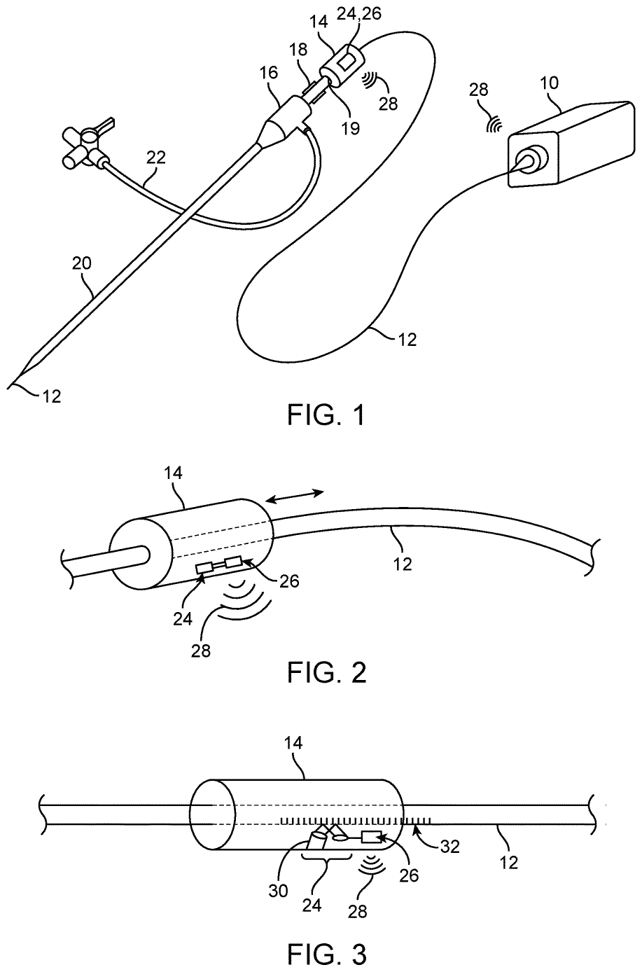

As shown in FIG. 1, an embodiment of a system according to the present disclosure includes patient interface module (PIM) 10, which may comprise an IVUS motor drive unit, and which includes a communication device (not shown) such as a Wi-Fi, custom radio, or Bluetooth module. IVUS catheter 12 extends from PIM 10 through sensing unit 14 and vascular introducer 16, which in some embodiments may be joined by a conventional Luer lock fitting 18 between the sensing unit and introducer. Sensing unit 14 may include a fitting 19 at one end connectable to Luer lock fitting 18. Catheter 12 extends from the distal end of introducer needle 20. Vascular introducer 16 also includes a saline/flush line 22 as is known in the art. Motion sensing unit 14 includes motion sensing element 24 and communications device 26, each of which is disposed with a housing of sensing unit 14, either on or within the housing. Communications device 26 produces wireless communications signal 28. Wireless communication signal 28 may be directed to and received by PIM 10, and also may be received by an IVUS console (not shown) that includes system processing and image display to the operator as is also known in the art.

Sensing element 24, which detects motion of the IVUS catheter as it is moved past the sensing element, such as by sliding or translating motion, may take a number of different forms. For example, as shown in FIG. 2, in one embodiment it comprises a linear motion sensor in combination with a 3-axis accelerometer. In other embodiments, it may comprise a linear motion sensor alone. Examples of linear motion sensors include mechanical sensors such as a wheel or encoded wheel in contact with the catheter that turns as the catheter moves longitudinally with rotations of the wheel being sensed optically or by other rotary position/sensing means as may be configured by persons of ordinary skill. Other known linear motion/position sensing devices such as magnetic, inductive, capacitive or eddy current sensing may be used in combination with appropriate sensing treatments in or on outer surface of catheter 12. Magnetic distance sensors convert magnetic energy to electronic signals. Inductive linear sensors would use an electromagnetic induction to detect displacement. Capacitive sensors would use a dielectric material to detect motion. Eddy current could utilize any metal sheet and it can detect any distance or changes to the metal sheet movement. The linear motion sensor of sensing element 24 is configured to detect the amount and direction of longitudinal movement of catheter 12 through sensing unit 14, for example during pullback in the creation of an IVUS image. In embodiments also employing a 3-axis accelerometer in sensing element 24, the longitudinal movement information is combined with 3-axis accelerometer information detecting 3D movements of sensing unit 14 to detect longitudinal catheter movements and potential sensing unit 14 displacements during the procedure.

In one example, as shown in FIG. 3, the linear sensor of sensing element 24 may comprise an optical emitter/detector pair 30 such as an optical sensor in an optical computer pointing device (e.g., mouse). In this embodiment, sensor 30 uses the catheter's surface texture as a guide for calculating displacement and is able to provide micrometer-level displacement information. In some embodiments, and as shown in FIG. 3, graduated marks 32 may be provided on catheter 12 to increase optical sensing sensitivity and accuracy and can be used for the correction of sudden slips. When included in sensing element 24 with optical sensor 30 the 3-axis accelerometer further collects information indicating the 3D movements of sensing unit 14. To improve the accuracy of the 3D motion sensing, this accelerometer may include a 3-axis gyroscope and a 3-axis compass. Such a 3-axis accelerometer may be configured as a MEMS device.

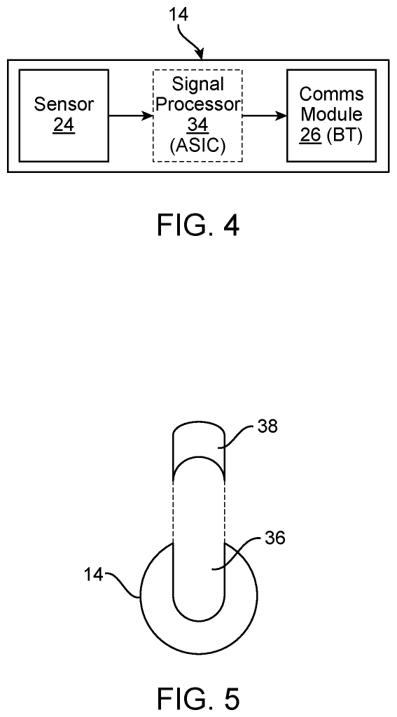

It may be desirable in some embodiments to include an optional signal processor 34 within motion sensing unit 14 as shown in FIG. 4. Processor 34 may, for example, be configured to amplify the output of sensing element 30 and digitize the signal where sensing element 30 produces an analog output signal. Processor 34 for example can be a Bluetooth® or Bluetooth® Low Energy (BLE) wireless microcontroller unit (MCU). In one embodiment, processor 34 may be executed as an ASIC by persons of ordinary skill based on the teachings herein. Processor 34 may also combine the information of all the sensors in unit 14 and extract the exact amount of catheter core and introducer needle displacements relative to each other, and to calculate an accurate displacement of the catheter core relative to the vessel. This information can be used to generate pullback length and in some embodiments may eliminate the need for a separate IVUS motorized pullback device. Alternatively, processor 34 can be eliminated, and raw linear displacement and 3D sensor information be transmitted as is. In this configuration, the raw information will be processed by the wireless system console (not shown) to generate the catheter displacement information noted above.

Sensing unit 14 may be configured as an inexpensive, disposable, add-on accessory device that can easily be adapted to an existing IVUS system simply by attaching a Luer lock fitting 18 before introduction of catheter 12. Use of sensing unit 14 allows for much greater control and more accurate knowledge of catheter position and pullback distance than is possible with current systems. Wireless communication signal 28 from communication device 26 may be directed to and received by interface unit/motor drive unit 10, and also may be received by a wireless system console (not shown), which includes system processing and image display to the operator. Sensing unit 14 also may be used in conjunction with a motorized pullback unit (not shown) with or without direct wireless communication of the wireless sensing signal 28 with the pullback unit. In some embodiments, it may be desirable to operate the motorized pullback unit under feedback control from sensing unit 14.

In a further alternative embodiment as shown in FIG. 5, sensing unit 14 may have a lengthwise opening 36 closeable by a cover or door 38 to allow the sensing unit to be placed onto and removed from catheter 12 without removing the catheter from the introducer. In another alternative, sensing unit 14 may be separate from vascular introducer 16 and Luer lock fitting 18 (or any other relatively fixed structure) and instead hand held by the operator when catheter 12 is pulled back. In the case of a handheld sensing unit 14, the acceleration sensor of sensing element 24 may be used to account for movements in the operator's hand that might otherwise affect the pull back measurement of the linear motion sensor.

The foregoing has been a detailed description of illustrative embodiments of the disclosure. It is noted that in the present specification and claims appended hereto, conjunctive language such as is used in the phrases “at least one of X, Y and Z” and “one or more of X, Y, and Z,” unless specifically stated or indicated otherwise, shall be taken to mean that each item in the conjunctive list can be present in any number exclusive of every other item in the list or in any number in combination with any or all other item(s) in the conjunctive list, each of which may also be present in any number. Applying this general rule, the conjunctive phrases in the foregoing examples in which the conjunctive list consists of X, Y, and Z shall each encompass: one or more of X; one or more of Y; one or more of Z; one or more of X and one or more of Y; one or more of Y and one or more of Z; one or more of X and one or more of Z; and one or more of X, one or more of Y and one or more of Z.

Various modifications and additions can be made without departing from the spirit and scope of this disclosure. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present disclosure. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this disclosure.

Exemplary embodiments have been disclosed above and illustrated in the accompanying drawings. It will be understood by those skilled in the art that various changes, omissions and additions may be made to that which is specifically disclosed herein without departing from the spirit and scope of the present disclosure.

1. A pullback distance sensor for intravascular ultrasound systems (IVUS), comprising:
a housing defining a central opening configured to slidingly receive an IVUS catheter therethrough;
a linear motion sensor disposed with the housing configured to detect motion of the IVUS catheter when moved through the housing central opening; and
a wireless communications device disposed with the housing in communication with the linear motion sensor to receive and wirelessly transmit signals representing movement of the IVUS catheter detected by the linear motion sensor.

2. The pullback distance sensor of claim 1, further comprising an acceleration sensor disposed with the housing in communication with the wireless communications device, wherein the acceleration sensor generates three-dimensional position information with respect to the housing to be wirelessly transmitted by the communications device.

3. The pullback distance sensor of claim 1, further comprising a connector fitting disposed at one end of said housing around the central opening to permit connection to a fitting or hub of an IVUS system introducer.

4. The pullback distance sensor of claim 1, wherein the housing further defines a lengthwise opening configured to allow placement of the housing over the IVUS catheter and further comprise a closure for said lengthwise opening.

5. The pullback distance sensor of claim 4, wherein the housing is configured as a handheld housing.

6. The pullback distance sensor of claim 1, wherein the linear motion sensor comprises a wheel configured to contact the IVUS catheter as it is pulled through the housing.

7. The pullback distance sensor of claim 1, wherein the linear motion sensor comprises an optical sensor configured to detect movement of a surface of the IVUS catheter as it is pulled through the housing.

8. The pullback distance sensor of claim 7, wherein the optical sensor comprises an emitter-detector pair.

9. The pullback distance sensor of claim 7, wherein graduated marks are disposed on the surface of the IVUS catheter.

10. The pullback distance sensor of claim 1, wherein the linear motion sensor comprises a magnetic linear position sensor and the IVUS catheter includes one or more magnetic elements along its length.

11. The pullback distance sensor of claim 1, wherein the linear motion sensor comprises an inductive linear position sensor and the IVUS catheter includes a magnetic core or magnetic elements along its length.

12. The pullback distance sensor of claim 1, wherein the linear motion sensor comprises a capacitive linear position sensor and the IVUS catheter includes dielectric material such as ceramic or glass along its length.

13. The pullback distance sensor of claim 1, wherein the linear motion sensor comprises an eddy current linear position sensor and the IVUS catheter includes a conductive metal strip or strip elements along its length.

14. The pullback distance sensor of claim 1, further comprising a signal processor disposed with the housing in communication with at least the linear motion sensor and wireless communications device, wherein said signal processor is configured to determine pullback distance of the IVUS catheter-based inputs from said sensors.

15. The pullback distance sensor of claim 14, wherein the signal processor is configured to receive acceleration sensing signals from the acceleration sensor to determine three-dimensional motion of said housing.

16. An intravascular ultrasound system (IVUS), comprising:
an IVUS patient interface module;
an IVUS catheter extending from the patient interface module;
an IVUS catheter introducer configured to receive the IVUS catheter;
a pullback distance sensor configured to be disposed around the IVUS catheter and produce a wireless signal indicating pullback distance, wherein said wireless signal is received by an IVUS system controller.

17. The intravascular ultrasound system (IVUS) of claim 16, wherein the IVUS system controller receiving said wireless signal comprises the IVUS patient interface module.

18. The intravascular ultrasound system (IVUS) of claim 17, wherein the IVUS system controller further comprises an IVUS console and said IVUS console additionally or alternatively receives said wireless signal.

19. The intravascular ultrasound system (IVUS) of claim 1, wherein the pullback distance sensor comprises:
a housing defining a central opening configured to slidingly receive the IVUS catheter therethrough;
a linear motion sensor disposed with the housing configured to detect motion of the IVUS catheter when moved through the housing central opening; and
a wireless communications device disposed with the housing in communication with the linear motion sensor to receive and wirelessly transmit signals representing movement of the IVUS catheter detected by the linear motion sensor.

20. The intravascular ultrasound system (IVUS) of claim 19, wherein the pullback distance sensor further comprises an acceleration sensor disposed with the housing in communication with the wireless communications device, wherein the acceleration sensor generates three-dimensional position information with respect to the housing to be wirelessly transmitted by the communications device.

21. The intravascular ultrasound system (IVUS) of claim 19, further comprising a connector fitting disposed at one end of said housing around the central opening to permit connection directly or indirectly to the IVUS catheter introducer.

22. The intravascular ultrasound system (IVUS) of claim 19, wherein the housing further defines a lengthwise opening configured to allow placement of the housing over the IVUS catheter and further comprises a closure for said lengthwise opening.

23. The intravascular ultrasound system (IVUS) of claim 22, wherein the pullback distance sensor is configured as a handheld unit.

24. The intravascular ultrasound system (IVUS) of claim 19, wherein the linear motion sensor comprises a wheel configured to contact the IVUS catheter as it is pulled through the housing.

25. The intravascular ultrasound system (IVUS) of claim 19, wherein the linear motion sensor comprises an optical sensor configured to detect movement of a surface of the IVUS catheter as it is pulled through the housing.

26. A method of measuring pullback distance in an intravascular ultrasound system (IVUS), comprising:
placing a pullback distance sensor housing around an IVUS catheter;
pulling the IVUS catheter through the pullback distance sensing unit;
detecting a distance of travel of the IVUS catheter through the pullback distance sensor housing with at least a linear motion sensor disposed with said unit housing; and
wirelessly transmitting a signal indicating said distance of travel from the pullback distance sensor housing to an IVUS system controller.

27. The method of claim 26, wherein the IVUS system controller comprises one or both of an IVUS console and an IVUS patient interface module.

28. The method of claim 26, further comprising attaching the pullback distance sensor housing directly or indirectly to an IVUS catheter introducer for placement of the IVUS catheter within a patient's vasculature.

29. The method of claim 26, further comprising holding the pullback distance sensor housing in a hand of a system operator during at least said pulling and detecting steps.

30. The method of claim 26, further comprising processing signals from at least the linear motion sensor within the pullback distance sensor housing to determine a pullback distance and wirelessly transmitting said determined pullback distance.

31. The method of claim 30, further comprising processing signals from a three-axis accelerometer included with the liner motion sensor to determine three-dimensional movement of the pullback distance sensor housing and optionally correct the determined pullback distance based on the determined three-dimensional movement.
